(19) United States
(12) Patent Application Publication
(10) Pub. No.: US 2009/0041823 A1
(43) Pub. Date: Feb. 12, 2009

(54) Agent-releasing Vascular Prosthesis

(76) Inventors: Axel Larena-Avellaneda, Sommershausen (DE); Rolf Siegel, Wurzburg (DE); Eike Sebastian Debus, Hamburg (DE)

Correspondence Address: DYKEMA GOSSETT PLLC, 10 S. WACKER DR., STE. 2300, CHICAGO, IL, 60606, US

(21) Appl. No.: 10/572,447

(22) PCT Filed: Sep. 18, 2004
(86) PCT No.: PCT/EP2004/010514

§ 371 Date: Oct. 6, 2008

(30) Foreign Application Priority Data
Sep. 19, 2003 (DE) 103 43 922.6
Nov. 20, 2003 (DE) 103 54 359.7

(51) Int. Cl. A61F 2/02; B05D 3/00

(52) U.S. Cl. 424/423; 427/2.24

(57) ABSTRACT

The invention relates to a prosthesis comprising a proximal and a distal end and a hollow space that extends there-between. Said prosthesis is provided with a support structure and at least one biologically active compound.

The present invention is related to a prosthesis and a method for the manufacture thereof.

Vascular prostheses are an important tool for the physician in modern therapy in connection with numerous diseases such as, for example, vascular sacculation, vascular stenosis or obstructions in case of arterial occlusive disease/diabetes mellitus and chronic renal failure, particularly if vascular reconstruction is necessary due to a disease and such vascular reconstruction using autologous vascular material is not possible as said vascular material is either pathologically altered or has already been removed. In the prior art plastic prostheses are used in such cases. Because of their application as vascular substitute it is preferred that vascular prostheses are primarily impermeable. The term vascular prostheses covers various moulded articles such as tubes, bifurcations grafts or patches which contact biological material. Tubes have a proximal and a distal end which are connected to each other by a lumen extending in between, whereby the lumen allows for containing a fluid, whereby the fluid cannot pass from the lumen through the wall of the prosthesis and whereby liquids from outside of the vascular prosthesis cannot pass into the lumen. Bifurcated grafts are Y-formed branched tubes consisting of two proximal ends and one distal end and which are linked by a lumen extending in between. The lumen allows for containing a liquid, whereby the liquid cannot pass from the lumen through the wall of the prosthesis and whereby the liquids cannot pass from outside of the vascular prosthesis into the lumen. Y-prostheses and tubes have two contact surfaces, namely an inner wall within the hollow body facing the blood, and an outer wall of the hollow body facing the tissue.

In contrast thereto, patches, also referred to as patch plasty do not comprise a lumen, but have a two-dimensional shape such as a flap and comprise two contact surfaces which get in touch with biological tissues.

The use of knitted polyester prostheses as vascular prostheses is described in the prior art. Such knitted polyester prostheses are primarily leaky. Accordingly, they are transformed into a form which eliminates such leakiness. This is typically performed by coating the polyester prosthesis with bovine collagen, albumin or gelatin which, however, is disadvantageous insofar as they introduce animal products into the human body which is, also because of regulatory reasons, increasingly less accepted with regard to bovine spongiform encephalopathy (BSE).

A further support material used in the prior art for prostheses is expanded polytetrafluoroethylene (PTFE) which, however, has a tendency to cause stitch track bleeding upon implantation which one tries to avoid by coating the materials with collagen, albumin or gelatin which goes along with the above described disadvantageous effects.

Furthermore, the vascular prostheses made of plastic which are described in the prior art, have in general the disadvantage that they are biologically not inert, i.e. they are degraded over time, that their surfaces are frequently thrombogenic and that they result in formation of intimal hyperplasia, in particular at the anastomoses.

Intimal hyperplasia is a reaction of the vasculature to an injury (Larena-Avellaneda et al. 2004, Weiterführende Modifikation und Entwicklung des “drug-eluting bypass”, Gefäβchirurgie, in press). The smooth muscle cells propagate in the media of the artery. They migrate into the intima and start producing so called extra-cellular matrix.

These disadvantages associated with the use of such kind of vascular prostheses are, apart from the progress of the underlying diseases which cause the implantation of such prostheses, are the main reason, why plastic prostheses according to the prior art do not have, from a medial point of view, the desired and required effect. Inertness means that the prosthesis is not degraded upon implantation. Thrombogenicity means the characteristic of a vascular prosthesis to induce the formation of a thrombus and/or its deposition upon contact of the same with blood. The progress of intimal hyperplasia is, according to the current understanding, based on an injury of the endothelial cells which results in the adsorption of thrombocytes. These as well as the vascular cells produce growth factors such as, PDGF, FGF-2, Endothelin I, Angiotensin II and matrix metalloproteinases [Bauters C. et al., Cardiovasc.Res. 31:835-846; Ben Slimane S. et al., Eur.Surg.Res. 20:12-17], which then, together with other factors such as, for example, hormones and mechanical factors, result in intimal hyperplasia by changes of the smooth muscle cells, whereby an activation of the smooth muscle cells can be detected already 30 minutes after vascular injury. 24 to 48 hours later the smooth muscle cells in the media start to proliferate. After four days the smooth muscle cells migrate into the intima and after one week the smooth muscle cells show a maximum proliferation rate. This goes on for some weeks, whereby additional extra-cellular matrix with collagen of collagen type I/III, elastin, fibronectin and proteoglycan are produced. A steady state is reached after about three months. The intima consists then of 47 to 80% of extra-cellular matrix. As a secondary effect monocytes, T-lymphocytes and other leukocytes from the plasma infiltrate the vascular wall.

Various approaches are described in the prior art in order to suppress intimal hyperplasia. These approaches comprise, for example, radiation of the anastomoses [Soni A. B. et al., Int.J. Radiat.Oncol.Biol.Phys. 54:1174-1179], gene therapeutical approaches [Conte M. S. et al., J. Vasc.Surg. 36:1040-1052] and tissue engineering [Teebken O. E. et al., Eur.J. Vasc.Endovasc.Surg. 23:475-485] as well as coating of the implant with endothelial cells [Arts C. H. et al., Eur.J. Vasc.Endovasc.Surg. 23:29-38; Conte M. S. et al., J. Vasc.Surg. 21:413-421; Gomes D. et al., J. Vasc.Surg. 34:707-715; Seifalian A. M. et al., Artif.Organs 26:307-320]. The coatings applied so far, such as carbon in case of the product Carboflo® of Impra, or Heparin in case of the product Propaten® of Gore did not show significant clinical success [Imig, H Grundmann, RT, 2000, Gefäβprothesen—Wo geht es hin?, Zentralbl. Chir. 125: 298). A decisive disadvantage of this kind of vascular prostheses is, for example, in the case of the product Propaten, the lack of local delivery of the drug Heparin which is bound to the prosthesis in an irreversible manner. Active sequences of the heparin molecules bound to the prosthesis protrude from the inner side of the prosthesis and bind anti-thrombin, whereby anti-thrombin has acquired such a conformation by the binding that it is capable of binding thrombin in a particular fast manner. The complex consisting of thrombin and anti-thrombin is washed away from the heparin molecule by the blood flow. By the formation of the complex the generation of the thrombus is to be avoided. Also in case of the product Carboflo® the drug Carbon is not released, but acts only as a bioactive coating. Also this surface coating is to render the prosthesis less thrombogenic.

However, all these approaches have in common that they are usually very material and time consuming and they are still at an early stage of their development, respectively, and their application is more of an experiment rather than daily clinical practice.

The problem underlying the present invention is thus to provide a prosthesis which overcomes the disadvantages of the prostheses of the prior art. In particular, a problem underlying the present invention is to provide an sealed prosthesis which remains significantly longer in the body after implantation compared to conventional prostheses. In accordance with the invention the prosthesis is biologically inert, shows little thrombogenicity and little tendency of forming an intimal hyperplasia upon implantation.

In a first aspect the problem underlying the present invention is solved by a prosthesis comprising a proximal and a distal end and a lumen extending in between, whereby the prosthesis comprises a support structure and at least one biologically active compound.

In an embodiment the support structure consists of a support material and a coating material.

In a preferred embodiment the support material is selected from the group comprising polytetrafluoroethylenes, polyesters and polyurethane.

In an embodiment the coating material is selected from the group comprising organopolysiloxanes.

In a preferred embodiment the organopolysiloxane is selected from the group comprising polydimethylsiloxane, polyvinylsiloxane, polyphenylsiloxane and polyalkylsiloxane.

In an embodiment the support material and the coating material form a matrix.

In a preferred embodiment the matrix contains the biologically active compound.

In an embodiment the support structure defines an outer wall and an inner wall, whereby the inner wall limits the lumen conducting the blood, and the outer wall limits the support structure against the surrounding tissue.

In a preferred embodiment the prosthesis is liquid tight.

In an even more preferred embodiment the coating material is polydimethylsiloxane and preferably the support material is polyester, whereby the coating of the prosthesis is 10-30 mg polydimethylsiloxane/cm² surface, preferably 15-26 mg polydimethylsiloxane/cm² surface and most preferably 16-23 mg polydimethylsiloxane per cm² surface.

In an embodiment the coating material comprises a modification.

In a preferred embodiment the modification is selected from the group comprising silanolate, alkyl hydroxide, polyvinyl alcohol, polyethylene oxide, zinc sulfate, polyvinyl pyrrolidone, and phosphocholine.

In a preferred embodiment the modification is selected from the group comprising polyvinyl alcohol, polyvinyl pyrrolidone and phosphocholine.

In an even more preferred embodiment the modification is present at the inner wall and preferably remains there permanently.

In an embodiment at least one biologically active compound is present in the matrix.

In an embodiment the biologically active compound is selected from the group comprising antibiotics, immunosuppressive agents and proliferation inhibitors.

In an embodiment the biologically active compound is selected from the group comprising acetylsalicylic acid, sirolimus and paclitaxel.

In an embodiment the biologically active compound is released at a rate of 2-3 μg/hour.

In an embodiment the prosthesis is a vascular prosthesis.

In an embodiment the prosthesis is used in alloplastic reconstruction extending beyond the knee.

In an embodiment the prosthesis is used as a peripheral or central AV shunt.

In an embodiment the prosthesis is used as a patch or as a dialysis shunt.

In a second aspect the problem underlying the present invention is solved by a method for the manufacture of a prosthesis comprising a proximal and a distal end and a lumen extending in between, whereby the prosthesis comprises a support structure and at least one biologically active compound and the support structure consists of a support material and a coating material, preferably a prosthesis according to the first aspect of the present invention, comprising the steps of:
providing the support material, whereby the support material comprises a proximal and a distal end and a lumen extending in between;
coating the support material with a coating material, whereby a matrix is formed; and
introducing the biologically active compound into the matrix.

In an embodiment of the second aspect of the present invention the coating of the support material is performed by applying the coating material onto the rotating support material.

In an embodiment of the second aspect of the present invention the coating step is repeated several times and, preferably, the applied coating material or the coated support material is dried after each coating step.

In an embodiment of the second aspect of the present invention the coating material already comprises a modification.

In an embodiment of the second aspect of the present invention the coating material and/or the matrix is provided with a modification, preferably on the surface, and more preferably at the wall of the support structure facing the lumen, and is modified therewith.

In a preferred embodiment of the second aspect of the present invention the modification is performed by a wet-chemical modification method.

In an embodiment of the second aspect of the present invention the modification is formed by a molecule or a group provided thereby which is selected from the group comprising polyvinyl alcohol, phosphocholine and polyvinyl pyrrolidone.

In a preferred embodiment of the second aspect of the present invention prior to the wet-chemical modification method the matrix is activated by means of an alkaline methanol solution.

In an even more preferred embodiment of the second aspect of the present invention after the activation the modification generating molecule is contacted with the matrix in a diluted solution, preferably contacted with the inner wall of the matrix and/or the support structure.

In an embodiment of the second aspect of the present invention the biologically active compound is introduced into the matrix by a wet-chemical method.

In a preferred embodiment of the second aspect of the present invention prior or during the introduction of the biologically active compound the matrix is swollen by contacting the matrix with a swelling agent.

In an embodiment of the second aspect of the present invention the biologically active compound is provided in a solvent and that the biologically active compound containing solvent is contacted with the matrix.

In a preferred embodiment of the second aspect of the present invention the solvent is a swelling agent for the matrix.

In an embodiment of the second aspect of the present invention the support material is rotated along its longitudinal axis which extends through or is parallel to the lumen of the support material while the coating material is applied to the support material.

In an embodiment of the second aspect of the present invention the coating material is provided as a pre-polymer and is polymerized during or after the contacting with the support material.

In a preferred embodiment of the second aspect of the present invention the coating material is applied from the distal end to the proximal end or from the proximal end to the distal end, whereby the support material is moved relative to the applied coating material in the direction of the longitudinal axis of the support material.

In an embodiment of the second aspect of the present invention the support material consists of a polyester and that the coating material is polydimethylsiloxane.

In a preferred embodiment of the second aspect of the present invention the support material is rotated at 80 revolutions/minute.

In an even more preferred embodiment of the second aspect of the present invention the coating material is a solution of 11 g of the pre-polymer of polydimethylsiloxane in 86 g ethylacetate.

In a particularly preferred embodiment of the second aspect of the present invention the relative movement is 14 mm/minute and the dripping rate is 15-20 ml/hour.

In an embodiment of the second aspect of the present invention a coating cycle is completed once the support material has been coated from its proximal end to its distal end or from its distal end to its proximal end.

In a preferred embodiment of the second aspect of the present invention the coating cycle is repeated several times.

In a third aspect the problem underlying the present invention is solved by a prosthesis obtainable by a method according to the second aspect of the present invention.

The present invention is based on the surprising finding that in case of a prosthesis comprising a support structure and at least one biologically active compound, thrombogenicity and intimal hyperplasia caused by such prosthesis can be reduced and avoided, respectively, at the same time. The support structure of the prosthesis of the present invention consists of a support material and a coating material. For avoiding the risk associated with the use of animal and in particular bovine material such as collagen, albumin or fibrinogen, as for example BSE, the coating material is preferably a plastic. The present inventor has surprisingly found that organopolysiloxanes are particularly suitable as plastics. By using this kind of plastic as a coating material, it is possible that various support materials of the prior art are used for the manufacture of prostheses or stents. A particularly preferred starting material is insofar polytetrafluoroethylene and polyesters such as, for example, polyethylene terephthalate, or polyurethane. It is within the present invention that the support material may in general be manufactured from such materials which can be used as blood conducting path.

The prosthesis of the present invention is multifunctional, i.e. by incorporating biologically active molecules onto the surface or into the matrix of the prosthesis they will exert local effects, i.e. for example optimum integration of the prosthesis with concomitant inflammation prophylaxis and/or thrombosis prophylaxis. The biologically active compound is preferably continuously released from the contact surfaces, both inside and outside, of the prostheses.

The prosthesis of the present invention is thus preferably a tubular vascular prosthesis. Apart from its use as a vascular prosthesis the prosthesis of the present invention can also be used for reconstruction of structures of the vascular type such as body passages or body ways.

In a preferred embodiment the prosthesis according to the invention is a moulded article for use as vascular substitute which is preferably used in connection with far reaching obstructions.

The prosthesis of the invention is a moulded article which has to contact surfaces with biological material. In a preferred embodiment the moulded article consists of a tube, i.e. a prolonged element having a proximal and a distal end and a lumen extending in between, which is also referred to herein as support structure. The lumen defines the region where the body fluid is to be guided after the introduction or implantation of the prosthesis. The flow direction of the fluid is essentially parallel to the longitudinal axis of the prosthesis defined by the lumen. The flow direction, however, can also depart therefrom, for example as in case of extra-anatomical derivation, coronary bypass and dialysis shunts. It is preferred that the liquid flow flows through the lumen and there is no passage over the prosthesis wall defining or limiting the lumen, at least no significant passage. The passage of the liquid flow is, however, allowed if the prosthesis of the invention is used as a peripheral or central AV shunt such as a dialysis shunt or a carotis patch. The prosthesis is thus formed by a support structure having a prosthesis wall, whereby the prosthesis wall defines an outer wall and an inner wall and the inner wall limits the lumen of the prosthesis. The prosthesis of the invention can also be referred to or used as bypass. The prosthesis of the invention can additionally be embodied as a patch, and thus have essentially a two dimensional structure, whereby it comprises two contact surfaces which contact a biological tissue. Due to the two dimensional design of the patch the support structure will form the two contact surfaces and the patch will not have any lumen as in case of the tubular or Y-shaped embodiments of the prosthesis of the invention.

Preferably, the prosthesis is a foreign body, i.e. a body which is different from the material present in the organism into which the prosthesis is implanted. Preferably, the prosthesis is different from an allogenic or autologous vascular implant.

In a further embodiment the support structure is defined by a fibrous or sheet-like material. In a further embodiment the support structure consists of a knitted or woven material.

The support structure comprises the support material and the coating material. The coating material is preferably an organopolysiloxane. Polyvinylsiloxane, polyphenylsiloxane and polyalkylsiloxane are particularly preferred members of the group of organopolysiloxanes. As used herein, the term alkyl refers to a straight or branched hydrocarbon chain which may comprise one or several substitutes. Preferably, the hydrocarbon chain does not comprise any substitutes. In a preferred embodiment the length of the alkyl chain is one to six carbon atoms, preferably one to four carbon atoms and more preferably one to two carbon atoms. A particularly preferred polyalkylsiloxane is polydimethylsiloxane, in particular addition cross-linking silicone types. The three dimensional cross-linking of linear polydimethylsiloxane chains results in a completed moulded article consisting of silicone. The coating of support materials or support structures with silicones and in particular with siloxanes is, for example, described by Rochow E., (Eugene Rochow, Silicon and Silicones, Springer Verlag, ISBN 3-540-17565-2).

The coating with the organopolysiloxane is an efficient way to render the support material fluid tight which per se is preferably not fluid tight. An assay for the assessment of fluid tightness is described in the example part. In accordance with the present invention a prosthesis is fluid tight at values of ≦5 ml/minute/cm² at a pressure within the vessel of 120 mmHG.

It is within the present invention that the support material and the coating material together form a matrix. It is, however, also within the present invention that the matrix is formed by the coating material or the coated support material.

The prosthesis of the present invention comprises in an embodiment one or several modifications. These modifications, without wishing to be bound thereto in the following, are particularly active in influencing the thrombogenicity of the prosthesis of the invention. The modification is preferably a modification of the matrix, preferably a surface modification of the matrix. As used herein, a surface modification is a modification which is present on a surface. Preferably the surface is the one in contact with the liquid contained in the lumen of the prosthesis or passing therethrough. Such a modification of the matrix, i.e. providing the matrix with one or several modifications, can be performed prior to the application of the coating material, i.e. prior to coating the support material with the coating material. In such case the coating material already bears the modification. Alternatively, a modification, particularly a modification of the matrix, can be performed after the support material had been coated with the coating material. The modification can be formed by the following molecules or the reactive groups provided by such molecules: alkyl hydroxide, polyvinyl alcohol (PVA), polyethylene oxide (PEO), zinc sulfate, silanolate, polyvinyl pyrrolidone (PVP) and phosphocholine (PC). Preferably the alkyl residue of the alkyl hydroxide is as generally defined herein for alkyl.

Polyvinyl alcohol (PVA), phosphocholine (PC) and PDMS surfaces have proven to be a modification having or causing particularly little thrombogenisity.

In an embodiment of the prosthesis of the invention the biologically active compound is contained in the matrix or is bound to the surface. It is within the present invention that the biologically active compound is present at least on one of the contact surfaces of the prosthesis and/or in the matrix, i.e. in the region of the support structure between the outer and the inner wall. It is within the present invention that the biologically active compound is present both at the inner wall, i.e. at the side of the support structure facing the lumen, as well as at the outer wall. It is particularly preferred the biologically active compound is contacted with the tissue surrounding the prosthesis after the implantation or such fluid and also contacted with the fluid contained in the lumen of the prosthesis and passing therethrough.

The biologically active compound is either covalently or in an ionic manner bound to the matrix. Preferably, the biologically active compound is immobilized to or within the matrix in an absorptive manner.

The biologically active compound is preferably selected from the group comprising antibiotics, immunosuppressive agents and proliferation inhibitors. In particular the use of immunosuppressive agents or proliferation inhibitors adds significantly to the suppression of undesired intimal hyperplasia. Particularly preferred are sirolimus and paclitaxel. Further preferred compounds are abciximab, estrogen, thrombocyte aggregation inhibitors such as ASS, vitamin K-antagonists such as coumarins, everolimus, tacrolimus and growth factors. It is within the present invention that also two or several biologically active compounds can be comprised by the prosthesis and are contained in particular within or on the matrix. The two or more biologically active compounds can be released at the same time or at different points in time. Examples therefore are in case of inflammatory processes an immunosuppressive agent and an antibiotic. Further preferred biologically active compounds are, for example, for thrombosis prophylaxis: thrombocyte aggregation inhibitors or vitamin K-antagonists or heparins, for improved functionality of the prosthesis: growth factors having local and peripheral effects, for inhibition of inflammation; corticosteroids and hormones such as estrogens, for the inhibition of hyperplasias monoclonal antibodies for inhibiting graft rejection reactions; immunomodulators such as cytokines or enzymes. Also, genetically modified cells and cell fragments, enzyme inhibiting proteins and peptides may be used. It is also possible to use retarding agents in order to cause delayed effects of the compounds. The agents can be chemically modified or be formulated. A retarded form of sirolimus is, for example, rapamycine-28-N,N-dimethylglycinate methanesulfonic acid. Coated stents having a slow eluting form of sirolimus, showed in studies a lower rate of intimal hyperplasia and restenosis compared to stents provided with a fast eluting form of sirolimus (Carter, A L et al., 2004, Cardiovascular Res., 63, 617-624).

Due to the mechanism on the generation of intimal hyperplasia as described in the introductory part which is incorporated herein by reference in order to avoid any unnecessary repetitions, it is preferred that a defined elution profile for the biologically active compound is realized, particularly with regard to the kinetic of the formation of the intimal hyperplasia. It is within the skills of the one of the art that by using different pharmaceutically or biologically active compounds a suitable elution profile is established for the individual compound of the prosthesis of the invention. It can be determined while taking into consideration the particularities of the individual case such as flow characteristics, extent of liquid flow, efficacy of the individual compound at the implantation site and the like. For example, in case of an immunosuppressive agent IS the delivery of the individual compound will be in the range of 1-10, preferably 2-3 μg/ml. It is within the present invention that the one skilled in the art of immobilization of biologically active compounds will perform the same such that a local delivery of the compound is effected. It is particularly preferred that a maximum efficient level of the biologically active compound is present locally and that the systemic level is below the minimum efficient doses so as to avoid side effects.

In accordance with the present invention the prosthesis of the invention is particularly used for alloplastic below knee or above knee reconstruction. The prosthesis can generally be used for peripheral as well as central AV shunts. Further applications of the prosthesis of the invention comprises its application as carotis patch, in particular a carotis patch after desobliteration such as ACVB, i.e. aortocoronar venous bypass or as an implanted dialysis shunt for dialysis treatment.

In accordance with a further aspect of the present invention a method for the manufacture of a prosthesis is provided, whereby the prosthesis is preferably a prosthesis as disclosed herein in the first aspect of the present invention. It will be acknowledged that both the features as well as the embodiments of the prosthesis as described in connection with the method of the present invention may also be features of the prosthesis in accordance with the first aspect of the present invention and vice versa, i.e. features and embodiments are individually and independent of the realization of the particular embodiments of the prosthesis described and disclosed herein in connection with the prosthesis of the invention in accordance with the first aspect of the present invention, also features and embodiments of the prosthesis obtained or obtainable by the method of the present invention in accordance with the second aspect of the present invention.

The method of the invention for the manufacture of such prosthesis comprises the following steps:
providing the support material, whereby the support material is a moulded article which is for vascular substitution,
coating the support material with a coating material, whereby a matrix is formed and,
introducing the biological active compound into the matrix or onto the surface.

In accordance with the first aspect of the present invention the support material can be a moulded article, whereby the moulded article is preferably selected from the group comprising tubes, Y-prostheses and patches.

Suitable support materials and coating materials are in particular those as disclosed herein in connection with the prosthesis of the invention. Although not limited thereto, the coating material is applied to the support material in connection with the present invention in particular by applying by dropping, whereby the support material rotates along its longitudinal axis which is defined by the lumen. The rotation is preferably a steady rotation which is advantageous insofar as an even coating in circumferential direction is ensured. The coating material used in accordance with the invention can be a coating material which already comprises the modifications as described in connection with the prosthesis of the invention. Alternatively, it is also possible that the coating material is provided with the modification after the coating of the support material. Accordingly, it is within the present invention that the modification is only introduced into the matrix.

In an embodiment of the method of the invention where the modifications are only introduced into the matrix, it is within the present invention that the biologically active compound is introduced into the matrix both after the modification of the matrix, i.e. after the incorporation of the modification(s) into the matrix, and prior thereto, i.e. that the biologically active compound is introduced into the matrix already prior to introducing the modification(s) into the matrix.

The coating process is preferably performed by using the coating material in its not completely polymerized form. It is contemplated that a pre-polymer or pre-form of the polymer is applied to the support material and that the formation of the polymer occurs either immediately after the application or delayed. The coating material can be applied to the support material in accordance with techniques known to the one skilled in the art. This comprises, among others, dipping methods, spraying methods or dripping methods, whereby dripping methods as described in more detail in the examples, are preferred. The dripping method is particularly useful for manufacturing circumstances which use liquid to tenacious coating materials.

It is within the present invention that the coating process may be repeated several times. Preferably, the end of the repetition of the coating process is reached when the prosthesis exhibits the desired tightness, the required wall strength, when the desired elasticity characteristics are shown by the prosthesis as needed for the intended individual use, and/or when the prosthesis is coated with the coating material to the extent that a degradation of the prosthesis in the organism into which the prosthesis is to be implanted, does or can no longer occur, or occurs or can occur with a significantly decreased rate.

It is within the present invention that the formation of the polymer occurs after each individual step, preferably by drying at preferably increased temperatures. Depending on the respectively used coating material, in particular the polyalkylsiloxane such as polydimethylsiloxane, temperatures ranging from 60 to 90° C., and preferably 75° C. are preferred, whereby the selection is within the skills of the ones of the art and, among others, takes into consideration the stability of the substances used. It will be acknowledged that coating agents which are cured based on a different polymerization mechanism, require corresponding other conditions which are, preferably, realized after each individual step.

The modification process, i.e. the process of introducing the modifications into the matrix, is preferably a wet-chemical process, whereby the compound providing the modifications and the molecule, respectively, corresponding thereto, are provided in a diluted solution. Preferably, the diluted solution is prepared by using a nonpolar solvent such as, for example, heptane or hexane as solvent.

Preferably, the matrix is activated prior to the contacting with the solution containing the molecules providing the modification. This activation acts such as to accordingly incorporate the modifications into the matrix or onto the surface. The activation occurs preferably by a basic methanol solution.

The biologically active compound contained in the prosthesis is preferably also introduced into the matrix by a wet-chemical process. Preferably, the biologically active compound is immobilized based on a physical binding mechanism.

In a preferred embodiment the biologically active compound is thus bound to the matrix, preferably to the coating material, in an adsorptive manner and can be eluted therefrom so that the prosthesis of the present invention is a prosthesis eluting the biologically active compound. It is advantageous for a particularly effective immobilization that the matrix is swollen prior to or during the introduction of the biologically active compound into the matrix. Depending on the support material and in particular on the coating material used, various swelling agents can be used for such purpose. Preferably, the biologically active compound is provided as a solution, whereby the solvent is preferably a or the swelling agent.

The invention is now further illustrated by reference to the following figures and examples from which further features, individually or in combination, embodiments and advantages of the invention may be taken.

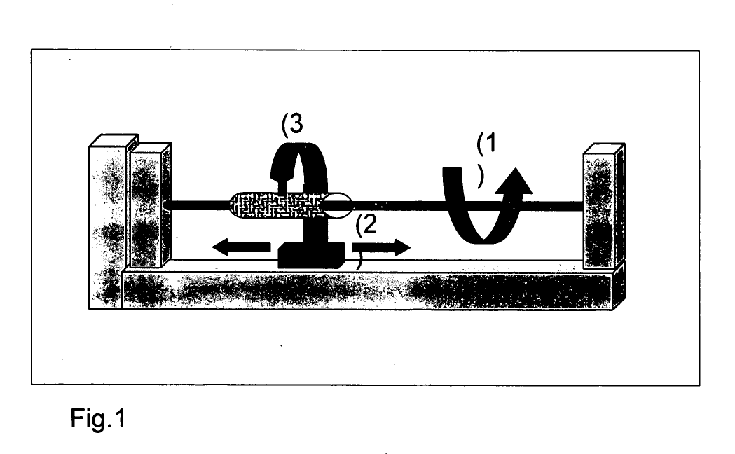

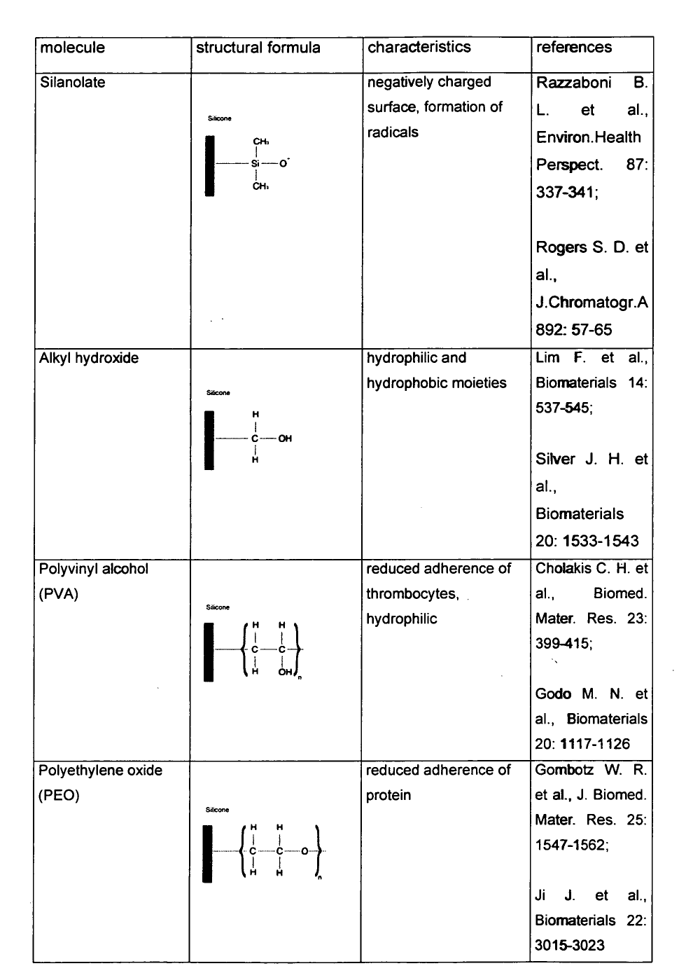

FIG. 1 shows a schematic representation of the process for manufacturing the prosthesis of the invention;

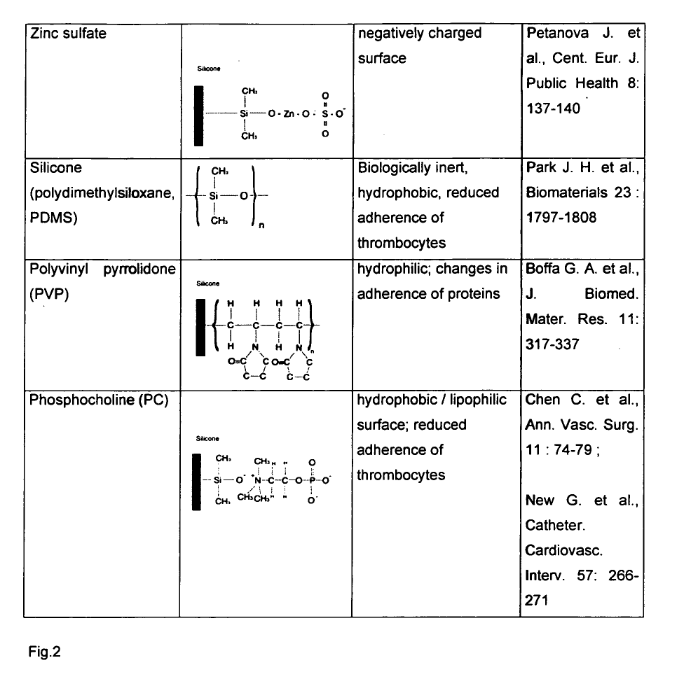

FIG. 2 shows the molecules used for surface modification which are also referred to herein as modification, whereby basic chemical structural elements which describe essential features with regard to thrombogenicity and references describing the respective modifications, are indicated;

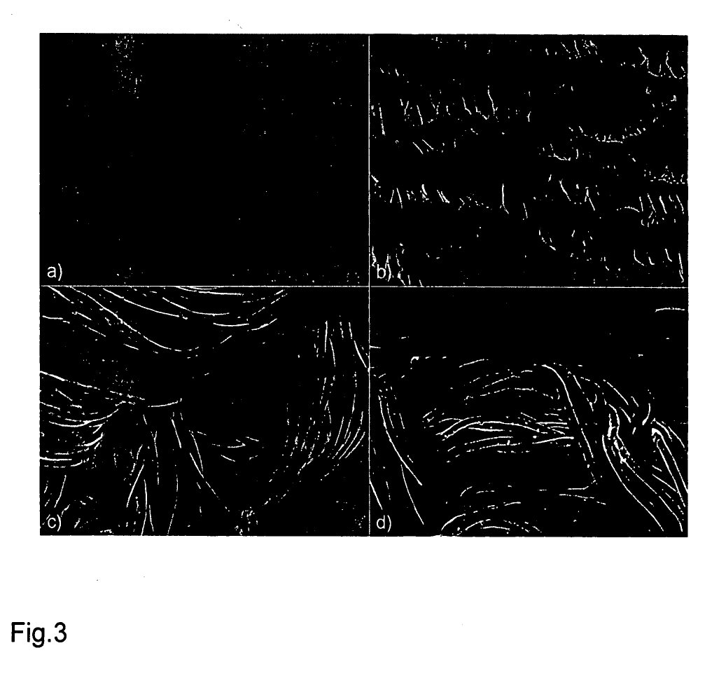

FIG. 3 is a scanning electron microscopic picture of various prostheses, whereby

FIG. 3 a) is a collagen coated polyester prosthesis of the prior art;

FIG. 3 b) is a PTFE prosthesis of the prior art;

FIG. 3 c) is a prosthesis of the present invention having a coating of 11 mg polydimethylsiloxane/cm² prosthesis surface; and

FIG. 3 d) is a prosthesis of the present invention having a coating of 20 mg polydimethylsiloxane/cm² prosthesis surface;

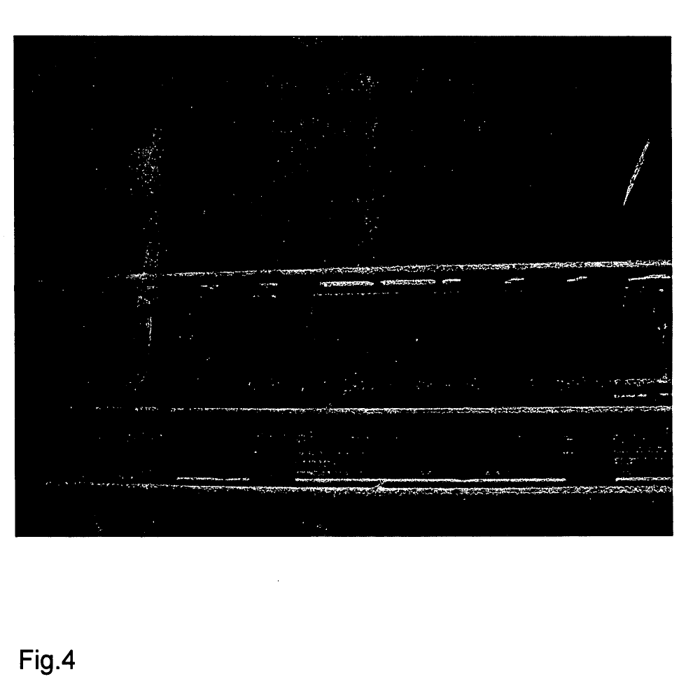

FIG. 4 depicts the blood distribution pattern in various surface-modified prosthesis after one hour of rotation, whereby the net-like distribution can be seen for prosthesis A (silonolate), E (zink sulfate), F (PDMS) and H (phosphocholine);

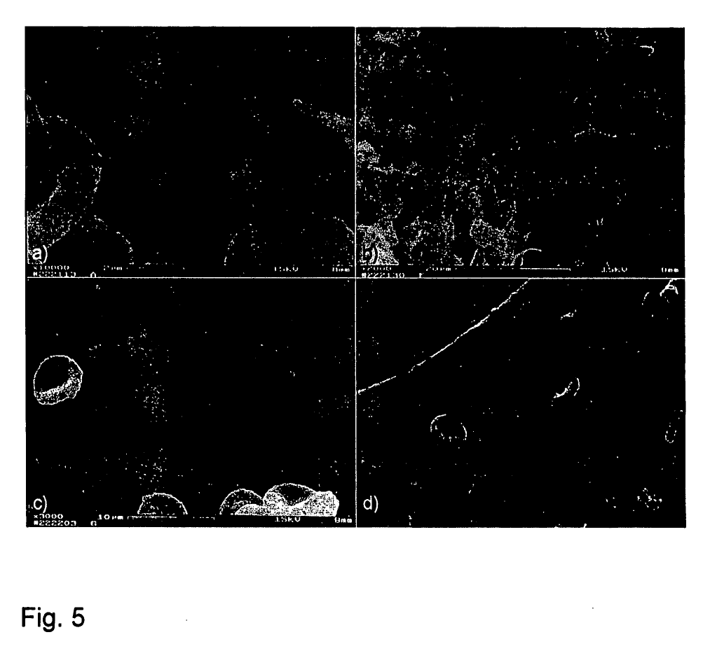

FIG. 5 shows a scanning electron microscopic picture of various prostheses after contacting blood;

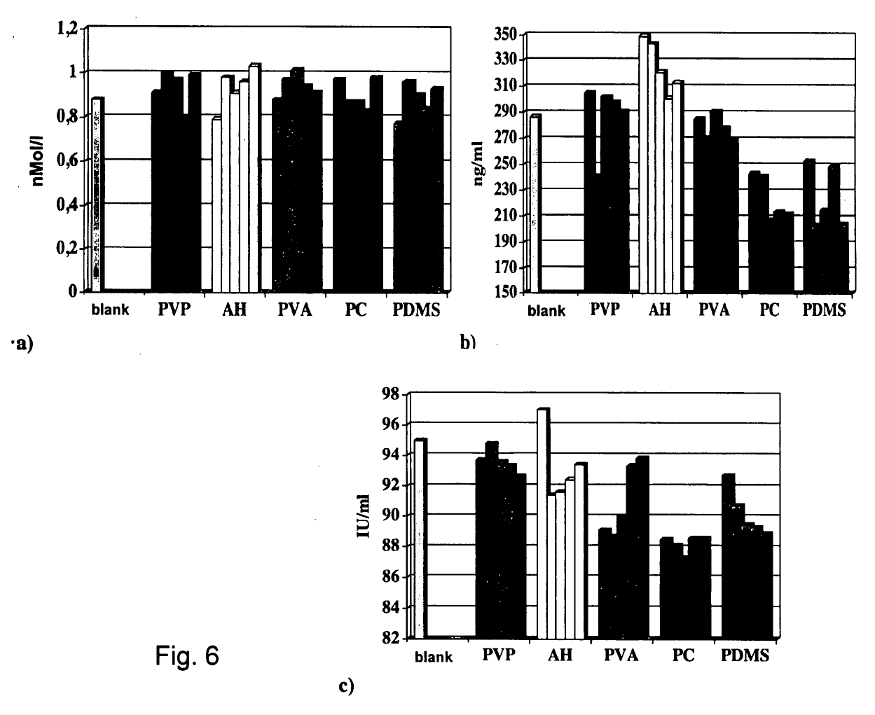

FIG. 6 shows the result of the determination of coagulation parameters upon use of various prostheses as determined in ELISA analyses;

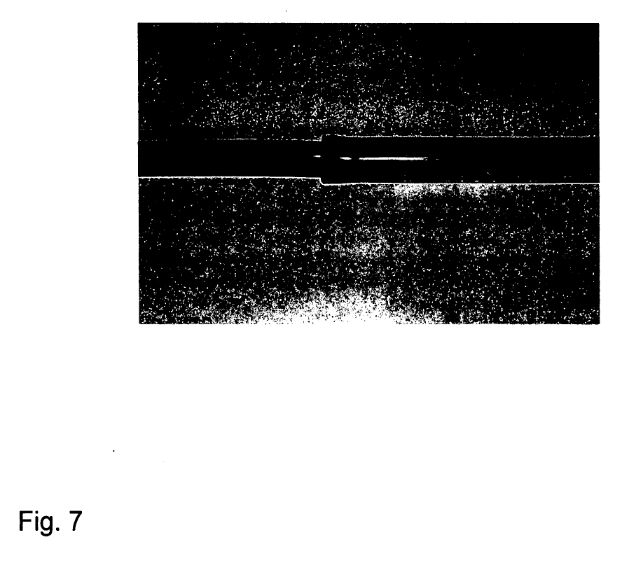

FIG. 7 shows the distribution of Sudan Red in the area of contact surfaces in connection with the examination of the elution of molecules in a circulation model;

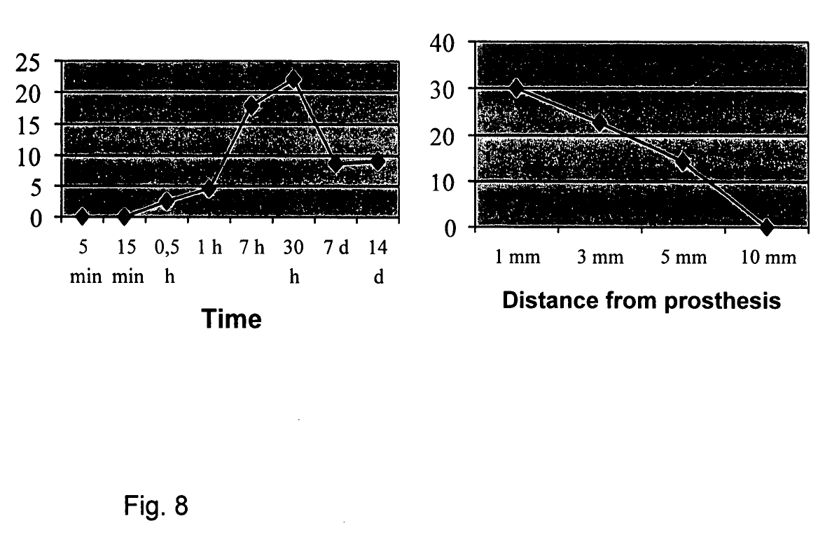

FIG. 8 a) shows a graphic representation of the release kinetics of the immunosuppressive agent IS in medium;

FIG. 8 b) shows the release of the immunosuppressive agent IS from a prosthesis of the present invention implanted into a bovine artery, as a function of the distance from the prosthesis;

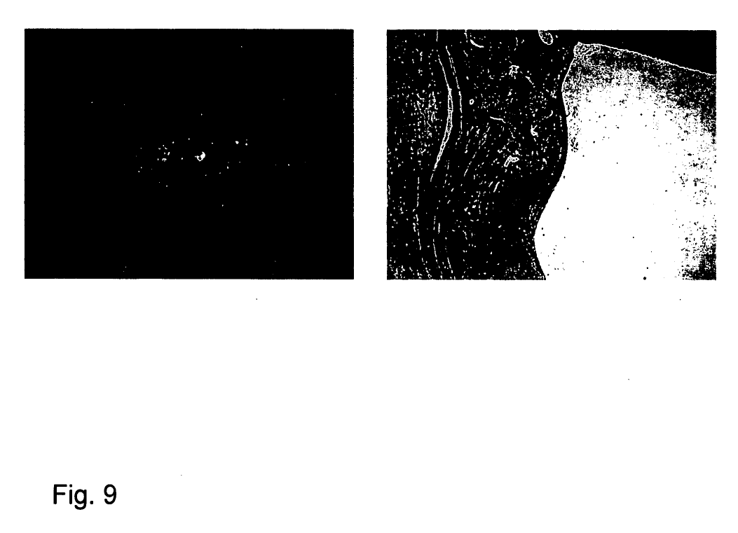

FIG. 9 a) is a photographic picture of an iliac bifurcation;

FIG. 9 b) is a histological representation of the prosthesis;

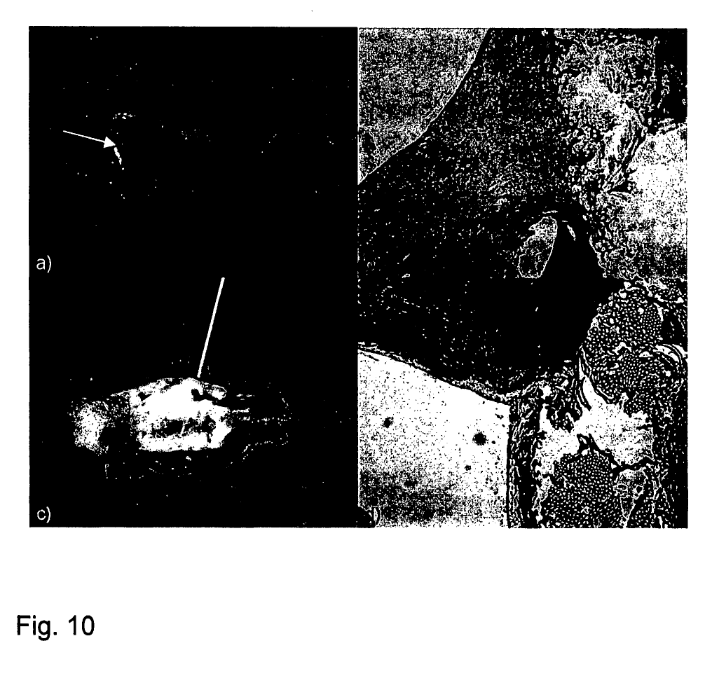

FIG. 10 shows an explanted iliac bifurcation together with prostheses according to the prior art (FIG. 10 a) and 10b)) and with prostheses of the present invention (FIG. 10 c) and d)); and

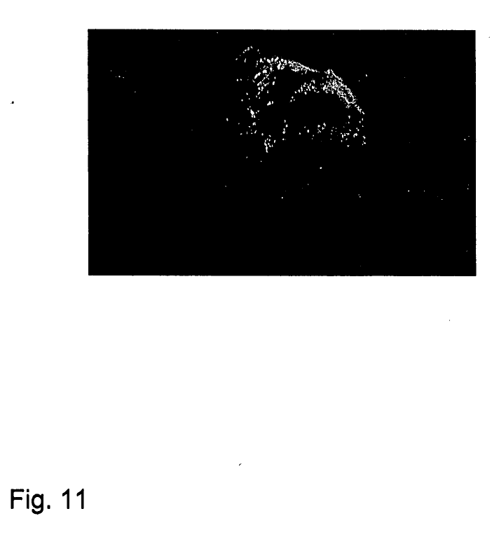

FIG. 11 shows a photographic picture of an explanted iliac bifurcation after implantation of a prosthesis of the present invention as patch plasty, whereby the prosthesis comprised paclitaxel.

### EXAMPLE 1

### Manufacture of a Prosthesis

A prosthesis having a diameter of 6 mm and a length of 200 mm consisting of knitted polyethylene terephthalate (Micron™, Intervascular), was provided. The prosthesis used as starting material is thus the support structure of the prosthesis of the present invention. The support structure was then transferred, as depicted in FIG. 1, onto a rotation rod by means of the lumen defining its longitudinal axis, and this rotation rod was mounted into a rotating production device. The rotation rod rotates at a defined speed around its own longitudinal axis (1). The production device depicted in FIG. 1 comprises a dosing device (3) which provides the coating means. In the present case the coating means is the pre-polymer of polydimethylsiloxane, namely MDX4-4210 of Dow Corning for animal trials, for use in human implant grade, from, for example, NuSil or Applied Silicone which was used as a solution of 11 g of the pre-polymer of polydimethylsiloxane in 86 g ethylacetate. The dosing device (3) is fixed in parallel to the longitudinal axis of the lumen and to the rotation rod (1) of the production device. The pre-polymer drops from the nozzle of the dosing device (3) onto the support material and coats the same. The pre-polymer is applied to the support material in circumferential direction by the support material being present on the rotation rod (1). Depending on the width of the outlet of the nozzle and the angle of dispersion and the angle of emergence thereof, respectively, a section of the circumference of the support material corresponding in width thereto, is coated with the pre-polymer. It will be obvious for the one skilled in the art that the width of the nozzle and the dropping rate will define the strength of the coating middle layer applied by the dripping. In the present case the width of the nozzle was 0.9 mm.

The manufacture of a prosthesis having a content of 20 to 22 mg silicone/cm² surface and having in particular the geometric surface of the support material was obtained by rotating the support material at 80 revolutions/minute. The advance of the dosing device (3) in direction (2) was 14 mm/minute. The dripping rate was 20 ml/hour in the first round of the coating. The process was repeated three times in total, whereby the dripping rate was 18 ml/hour in the second cycle of the coating and 17 ml/hour in the third cycle of the coating. Between the individual coatings the silicone was heated to 75° C. for curing.

In a second step the surface modification was introduced in a wet-chemical way. In principle, the modifications shown in FIG. 2 are suitable for being introduced onto and into the matrix formed by the support material and the coating material.

The results which were obtained with the various modifications, will be discussed individually in the following examples.

The surface modification was made such that the matrix was activated by treatment with basic methanol solution (5.7% by weight KOH for 12 minutes) and several washings with de-ionized water, subsequent treatment with 0.5% by weight PVP-solution (Luniquat PQ 11 PN of BASF, Leverkusen) for 120 minutes and subsequently by treatment with several washes. Thereafter, diluted solutions of the various molecules depicted in FIG. 2 were used in order to contact the thus activated matrix and to introduce the modification to the matrix.

In a further step the biologically active compound was introduced. Compounds such as, for example, ASS, gentamicin, paclitacel, sirolimus, an immunosuppressive agent IS are physically incorporated into the matrix of the silicone by contacting the prosthesis coated with cured silicone for 15 hours with a 0.05% by weight acetone solution of the active compounds, subsequently washing with acetone and drying over night at room temperature, whereby the silicone layer which was swollen due to acetone, returns to its original form. The process is an adsorptive process which allows the subsequent release of the biologically active compound after the implantation of the prosthesis.

The prostheses were plasma-sterilized and packed in a sterile manner for application.

### EXAMPLE 2

### Technical Auxiliary Means Used

For the various tests which are subject to the following examples the following technical auxiliary means were used. It is obvious for the one skilled in the art that similar instruments can, in principle, be used.

The weight of the prostheses for calculating the silicone content was determined by the use of a precision balance. The scanning electron microscopic assessment was performed after usual preparation in the “Zentrum für Elektronenmikroskopie” of University of Würzburg at a magnification of 50- to 5000-times. The examination on stability/elasticity were performed in a tensile testing machine and a cable tension meter, (Instron 4502, clamping yaw distance 2 cm, pull/push speed 50 mm/min) respectively, the force-path-diagrams were prepared by using a computer (Software Series IX, Instron). In order to determine the bursting pressure a balloon catheter (Ultrathin™Diamond™, 12 mm, Boston-Scientific) was used together with an inflator/manometer (Medflator II, Medex Medical, Lancashire, GB). The permeability and compliance measurements were performed by using a circulation model. A circulation consisting of 8 mm silicone tubes (Rehau, Rehau), was used as a basis. The model was used either in a closed or in an open form having two inlets/outlets. A circulation was established by a turbo-pump (12 V pump, Comet). Pressure and flow were continuously measured (manometer: GMH 3110, Greisinger, flow meter: DFM/POM+ARS 260 Totaliser, B.I.O. Tech). As a standard a flow of 500 ml/min at a pressure of 120 mm Hg was set. Furthermore, the precise inner diameter of the graft could be determined using an intravascular ultrasound device (IVUS; device: Clearview™Ultra, kindly provided by Boston Scientific, probe: Sonicath™Ultra 20 MHz, 5F, Meditech, Boston Scientific). Water was used as medium, to the extent necessary, in all tests for physical characteristics. Following the ISO standard, all analyses except the compliance measurement (37.2° C.) were performed at room temperature.

### EXAMPLE 3

### Physical Characteristics of the Prosthesis

The results of the determination of the physical characteristics of the prosthesis manufactured in accordance with example 1, can be summarized such that the prosthesis is not rendered instable by the silicone coating. Rather the advantage of the prosthesis of the present invention is, as also confirmed by experiments, that reactive molecules are masked at the surface of the support structure and that the prosthesis is not degradable upon implementation because of the silicone coating.

The silicone coated prosthesis as manufactured in example 1, was studied in more detail with regard to its physical and mechanical characteristics. In addition to the tests prescribed by ISO standard 7198 (see table 2) the prostheses were assessed from a subjective point of view, studied by scanning electron microscopy and the transversal and longitudinal elasticity as well as the puncture resistance were determined. Apart from non-coated Micron™prostheses primarily impermeable 6 mm implants consisting of PTFE (Goretex®, Gore) as well as collagen coated polyester (Intergard®, knitted, Intervascular/Meadox knitted Hemashield®, Boston Scientific) were used for comparison. In each case 3 prostheses were examined also following the ISO instructions.

The following table 2 sets forth the measured mechanical characteristics in accordance with ISO 7198: 2-9, the instruments used, the physical basics and the dimensions used. In connection therewith the following abbreviations are used:

Compliance: D=starting diameter of the prosthesis, ΔD the change of the diameter upon change of the pressure (ΔP). Elasticity: the law of Hooke is applicable to elastic deformation. F=acting force, E=elasticity (characteristic for the respective material). L=starting length of the relaxed prosthesis, Δl=change in length

TABLE 2

| quantity to be | | physical | |
|---|---|---|---|
| measured | device/medium | basics/formula | dimension |
| silicone content per | balance | weight (coated | mg/cm2 |
| cm² | | prosthesis)/cm² − | |
| | | weight (empty | |
| | | prosthesis)/cm² | |
| water permeability at | circulation model, | amount of water | ml/cm²/min at 120 |
| 120 mmHg | distilled water, | exited/surface of the | mm Hg |
| | measuring vessel | prosthesis | |
| stability: circular | tensiometer | Tmax/2L | kN/mm |
| stability: longitudinal | tensiometer | Tmax | kN |
| bursting pressure | balloon catheter | max. pressure | MPa |
| relaxed inner diameter | circulation model, | diameter of filled | mm |
| | IVUS, water | prosthesis | |
| Diameter at 120 mm | circulation model, | diameter at 120 mm | mm |
| Hg | IVUS, water | Hg | |
| compliance(dilatability) | circulation model,IVUS, water | $C = \frac{ΔD}{D \times ΔP} \times 100 \%$ | %/mmHg * 10⁻² |
| elasticity: longitudinal | tensiometer | $\begin{matrix}law of Hooke \\ F = E \times \frac{Δ l}{l}\end{matrix}$ | N |
| elasticity: transversal | tensiometer | law of Hooke | N |
| punctual resistance | tensiometer, straight needle | Force required to | N |
| | (Maxon 2x0, needle GR65) | penetrate material | |

The physical characteristics of the prosthesis can be summarized as follows.

Silicone Content:

Depending on the number of coatings the silicone content and the wall strength increase. The values, however, are within the range of conventional prostheses (table 3).

TABLE 3
Weight and wall strength of various 6 mm-prostheses per cm² surface.

| | wall strength | weight | silicone content |
|---|---|---|---|
| coating: | (mm) | (mg/cm²⁾) | (mg/cm²) |
| uncoated | 0.31 | 15.5 | |
| 1 × silicone | 0.33 | 26.6 | 11.1 |
| 2 × silicone | 0.35 | 32.1 | 16.6 |
| 3 × silicone | 0.43 | 38.1 | 22.6 |
| 4 × silicone | 0.46 | 41.4 | 25.9 |
| Meadox Hemashield ® | 0.40 | 18.4 | |
| Intergard knitted ® | 0.35 | 28.9 | |

Subjective Assessment:

The siliconized polyester prosthesis differs, depending on the amount of silicone, in its handling. Starting from a silicone content of 30 mg of silicone per cm² it impresses as comparatively stiff. On the other hand, an increased stability can thus be obtained in particular regions. Having one or two coatings (up to 20 mg/cm²) the prosthesis corresponds, from a subjective point of view, roughly to a common implant.

Scanning Electron Microscopy:

An even distribution of the silicone could be detected in scanning electron microscopy (FIG. 3). When the coating steps were repeated, a similar even coating with PDMS was observed.

Studies in the Tensile Testing Machine (Stability/Elasticity):

The stability as well as elasticity in length and transversal direction was determined as well as the puncture resistance. The results are set forth individually in the table. It can be taken therefrom that the stability of the prosthesis is not changed due to the silicone coating. The force which is required to disrupt the material is neither changed in longitudinal nor transversal direction due to the coating.

In contrast thereto, stiffness increases with the amount of silicone, in particular in transversal direction, although, to a lesser extent, also in longitudinal direction. The observed force results from the multiplication of the dilatation with the elasticity. As the dilation is known and the force is measured, this modulus can be determined for any of the material examined using the formula of Hooke for elastic deformation.

The stiffness in transversal direction correlates directly with the amount of silicone (table 4). It is higher for the prosthesis coated 2-, 3- and 4-times with silicone compared to the other materials. The stiffness in longitudinal direction depends on the crimping of the material. As the used empty prosthesis exhibits a comparatively strong crimping, the corresponding elasticity modulus is low. Insofar this result can not be compared to the results for conventional prostheses which have a less extensive crimping. This value increases less with the amount of silicone; obviously, silicone predominantly increases stability in transversal direction. The force which is required to penetrate the prosthesis (“puncture resistance”) is surprisingly for the PTFE prosthesis the lowest one and also increases with the extent of siliconization. Studies in our own clinic have shown that when a needle penetrates, a difference of >0.5 N can be sensed in a subjective manner.

TABLE 4

| | stiffness in | stiffness in | | |
|---|---|---|---|---|
| | transversal | longitudinal | | |
| | direction/elasticity | direction/elasticity | | |
| | modulus in | modulus in | | |
| | transversal direction | longitudinal | puncture | bursting pressure |
| material | (N/mm²) | direction | resistance (N) | (atm) |
| non-coated | 0.58 | 0.14 | 1.62 | >20 |
| 1 × silicone | 0.93 | 0.21 | 1.64 | >20 |
| 2 × silicone | 1.16 | 0.23 | 1.93 | >20 |
| 3 × silicone | 2.49 | 0.25 | 1.50 | >20 |
| 4 × silicone | 3.19 | 0.27 | 2.21 | >20 |
| Intergard ® | 0.8 | 0.54 | 1.87 | >20 |
| knitted | | | | |
| PTFE | 0.98 | 11.91 | 1.36 | >20 |

Circulation Model

Compliance and tightness were tested in the circulation model. The results are shown in table 5. The non-coated Micron™ prosthesis looses more than 1000 ml per minute already at low pressure. However, already a single coating round results in a sufficient tightness. The loss of water is 3 ml/min/cm² at 16-30 mg silicone/cm² and is thus within the range of conventionally used prostheses (table 5). Prostheses coated 3- and 4-times are completely tight.

The diameter of the prostheses in the relaxed form does not change due to the coating. However, it increases upon increasing siliconization at 120 mm Hg, so that a less dilatability, i.e. compliance, results. Also in relation thereto the results for the prostheses coated with 20-30 mg silicone are within the conventional range. As expected, the PTFE prosthesis shows hardly any change in diameter and thus a lower compliance.

TABLE 5
Physical characteristics of the studied materials. The following is
set forth: water permeability and compliance of the studied protheses

| | liquid loss | compliance |
|---|---|---|
| material | (ml/cm² * min) | (%/mmHg * 10⁻²) |
| not coated | >1000 | not examined |
| 1 × silicone | 680 | not e× amined |
| 2 × silicone | 3.7 | 6.29 |
| 3 × silicone | 0 | 5.29 |
| 4 × silicone | 0 | 4.03 |
| Meadox Hemashield ® | 2.9 | 5.77 |
| PTFE | 0 | 1.43 |

Bursting Pressure:

It was not possible to disrupt the polyester prosthesis using a 12 mm balloon catheter. The coating on the knitted structure, however, showed ruptures in longitudinal direction from 4-5 atm on. After maximum pressure inflation up to 20 atm, however, the prostheses had lost their elasticity and maintained a diameter of 12 mm. The amount of silicone did not have an impact on the bursting pressure (table 4). Also, the PTFE prosthesis did not disrupt.

### EXAMPLE 4

### Determining the Biological Characteristics of the Prosthesis

In order to assess the thrombogenicity of the various surface modifications some direct and some indirect detection methods were used. A direct assessment was performed by microscopy and scanning electron microscopy, respectively. Indirect detection was performed by determining the blood and coagulation parameters.

In order to simulate a sufficient surface contacting a model was established. For such purpose blood filled prostheses were rotated in transversal direction relative to the longitudinal axis for two hours at 37° C. The speed was selected so that an air bubble was running from one end of the tube to the other. Thus the whole surface was contacted in each prosthesis. The blood in all experiments was taken from the same donor, whereby anti-coagulation was performed with 3000 iU Heparin i.v. 30 minutes prior to blood sampling.

Macroscopic assessment of flow characteristics: In a first test series transparent silicone tubes were used. The different characteristics with regard to surface wetting were registered.

Scanning electron microscopic assessment: The prostheses were examined by scanning electron microscopy prior and after contact with blood.

General coagulation parameters: In the first experiments the following parameters were analyzed: Quick value, pTT, TZ, AT III, Fibrinogen, Hb, leukocyte number, thrombocyte number.

ELISA analysis of coagulation activators/of fibrinolysis as well as activation of thrombocytes: The blood was subsequently analyzed in more detail after having contacted the surface. First, the samples were centrifuged and thrombocyte-deprived citrate plasma was stored at −20° C. and later analyzed by means of the ELISA method.

Coagulation activation was detected by means of the prothrombin fragment F1+2 which is a cleavage product obtained in the formation of thrombin (used kit: Enzygnost® F1+2 Micro, Fa. Dade-Behring). D-Dimers are regarded as means for detecting the activation of fibrinolysis (Asserachrom® D-Dimer, Fa. Roche-Diagnostics). A further differentiation of the coagulation was performed with regard to endogenous=intrinsic and exogenous=extrinsic activation, respectively, (Marker: Imubind® Factor XIIa, Fa. American diagnostica and Tissue Factor (Imubind® Tissue factor, Fa. American diagnostica), respectively.

The detection of thrombocyte activation was performed by using the protein “β-thromboglobulin” (Asserachrom® β-TG, Fa. Roche-Diagnostics). This substance can be found in α-granula of the thrombocytes and is released therefrom upon their activation. The titer correlates with the degree of activation.

Macroscopic assessment of the flow characteristics: When assessing the flow characteristics of blood, characteristic changes were observed for the following molecules: hydrophobic substances resulted in a net-like, jerky flow (see FIG. 4, table 6), whereas hydrophilic molecules resulted in a smooth flow.

While no blood constituents adsorbed in case of PVA, PDMS and PVP, the surface of the prosthesis did not clear for the other materials. A partial thrombosis was observed in case of silanolate, PEO, zinc sulfate and phosphocholine, and in case of alkyl hydroxide wall thrombae were formed. The comparative polyester prosthesis had minimum wall thrombae.

TABLE 6
Thrombus formation in the various materials after one hour of rotation.

| material | formation of thrombus | flow characteristics |
|---|---|---|
| A - silanolate | partial thrombus | net-like |
| B - alkyl hydroxide | wall thrombus | smooth |
| C - PVA | no thrombus | very smooth |
| D - PEO | partial thrombus | jerky |
| E - zinc sulfate | partial thrombus | net-like |
| F - PDMS | no thrombus | net-like |
| G - PVP | no thrombus | very smooth |
| H - PC | partial thrombus | net-like |
| polyester coated | minimum wall thrombae | could not be |
| | | assessed |

The alkyl hydroxide is in particular ethanol.

Scanning electron microscopic assessment: Different characteristics were also observed at a magnification of 50 to 10,000 as shown in FIGS. 5 a)-d), whereby FIG. 5 a) shows the silanolate prosthesis with adsorbed erythrocytes at a magnification of 10,000, FIG. 5 b) shows a significant thrombus including fibrinogen net on the zinc surface at a magnification of 2,000, FIG. 5 c) is a representation of the PVP surface at a magnification of 3,000, where erythrocytes are loosely adsorbed without being adhered thereto, whereby the stripes in longitudinal direction are artifacts in the manufacture of the prosthesis; and FIG. 5 d) is a 1,000-times magnification of an albumin-coated polyester prosthesis of the prior art to which proteins and erythrocytes adhere.

General coagulation parameters: A further analysis was performed based on standard coagulation parameters. Prior to the administration of heparin standard values were observed, PTT of the subject was increased, as expected, upon administration of 3,000 iU Heparin i.v. After a contact time of 60 minutes this time was reduced for all prostheses. The best values were shown by alkyl hydroxide, PVA, PDMS, PVP and PC as well as coated polyester prostheses. No drop in thrombocyte and erythrocyte figures was found in connection with any of the materials. At first the values were determined at defined points in time after rotation had started (kinetics). For all of the materials a rapid decrease of PTT and TZ immediately after surface contact was observed. However, these values remained stable for the further course. The quick value was not influenced.

ELISA analysis: After determining the general coagulation parameter silanolate, zinc sulfate and PEO were excluded from further studies. In the ELISA studies a differing coagulation activation and fibrinolysis was shown for the prostheses.

We have found that the mere silicone tubes (i.e. the PDMS surface) and the PC coating as well as the PVA coating have a beneficial impact on coagulation inclination compared to other polyester prostheses. Other molecules in turn resulted in an increased coagulation inclination. This was confirmed in macroscopic, scanning electron microscopic and laboratory values (table 7).

TABLE 7
ELISA analysis after surface contacting. PC and PDMS exhibited the best values (4
experiments always two samples after 60 minutes of surface contacting). Average values as
well as standard deviations are indicated for prothrombin F1&2 (marker for coagulation
activation), D-Dimers (marker for fibrinolysis activity) and β-thromboglobulin (thrombocytes
activation).

| surface/parameter | blank | PVP | AH | PVA | PC | PDMS |
|---|---|---|---|---|---|---|
| prothrombin F1&2 | 0.88 | 0.80 ± 0.08 | 0.96 ± 0.23 | 0.95 ± 0.28 | 0.84 ± 0.28 | 0.83 ± 0.22 |
| (nMol/l) | | | | | | |
| D-Dimers | 286.5 | 299 ± 43.1 | 301 ± 7.1 | 278 ± 19.1 | 214 ± 36.8 | 249 ± 49.5 |
| (ng/ml) | | | | | | |
| β-thromboglobulin | 95 | 93.3 ± 17.3 | 92.4 ± 13.6 | 93.3 ± 17.3 | 88.6 ± 5.0 | 89.3 ± 10.0 |
| (IU/ml) | | | | | | |

There were no consistent results with regard to kinetics. PVP and PC showed an increase in prothrombin F1+2 immediately after the contact which is an indication for the activation of coagulation. However, in the further course these values decreased (FIG. 6 a)). In contrast thereto, alkyl hydroxide, PVA and PDMS did neither show an increase nor a decrease in the course, whereby the results from ELISA analysis are depicted in FIG. 6, indicated 5, 15, 30, 60 and 120 minutes after the beginning of rotation, whereby FIG. 6 a) depicts prothrombin F1+2, FIG. 6 b) D-Dimers and FIG. 6 c) β-thromboglobulin and the various surfaces are referred to as follows:

PVP=polyvinyl pyrrolidone;

AH=alkyl hydroxide;

PVA=polyvinyl alcohol;

PC=phosphocholine;

PDMS=polydimethylsiloxane.

For the D-Dimers and thus the activation of fibrinolysis there was a clear increase for alkyl hydroxide immediately after surface contact (FIG. 6 b)). β-thromboglobulin and thus the activation of the thrombocytes was decreased for PVA (however with increasing level over time), for PDMS and in particular for PC (FIG. 6 c)). For tissue factor and factor XIIa (both not represented) no significant tendency with the exception of single runaways could be found; there were standard values in nearly all of the measurements for all prostheses.

### EXAMPLE 5

### Elution of Molecules from the Prosthesis—Studies in the Circulation Model

The elution of molecules was first detected in the circulation model and then detected ex-vivo (bovine artery). The following substances were studied: acetylsalicylic acid (ASS), Sudan Red, gentamicin, sirolimus and immunosuppressive agent IS. Human albumin 5% was used as medium at room temperature.

In order to test for the wash out of ASS the pH of the medium was determined at regular times and thus a semi-quantitative assessment obtained. Sudan Red which is a hydrophobic dye, was analysed by photometry. The peaks for photometry are at wavelengths of 375 and 540 nm. sirolimus and IS were detected by quantitative titer determination (Labor Limbach, Heidelberg as well as Zentrallabor, Universitätsklinik Würzburg). For ex-vivo analysis the multifunctional prosthesis was anastomosed with a bovine artery (4 mm, Procol®, Fa. LA Med) and implanted into the circulation model. After 24 hours wall pieces were cut out from the artery in defined distances to the anastomosis, homogenized and centrifuged. Sirolimus and IS levels, respectively, were measured in the supernatant.

The first prostheses contained ASS (acetylsalicylic acid) in different concentrations. Based on the decrease in pH in the medium by two units within one week we could detect gradual elution of this hydrophilic, acidic molecule.

In order to test the elution of a hydrophobic molecule Sudan Red was incorporated into the prosthesis, whereby the distribution of Sudan Red in the region of the contact surfaces is shown in FIG. 7. However, at no time this dye could be detected by photometry in the liquid of regularly taken samples. Rather, the adjacent tubes incorporated the dye, in flow direction significantly more as in the opposite direction. The delivery of the hydrophobic agent into the surrounding tissue could thus be evidenced.

In a next step biologically active compounds were introduced into the prosthesis. The immunosuppressive agent IS was gradually released from the prostheses into the medium, whereby the release of the immunosuppressive agent IS into the medium is depicted in FIG. 8 a) and in the adjacent bovine artery in FIG. 8 b). The compound could be detected in the medium for the first time after 30 minutes and reached a maximum after 30 hours. Based on the amount of circulating medium we could calculate a release of 2-3 μg IS per hour. In the further course the concentration decreased.

After one week the level in the medium was 8-9 μg/l. After exchanging the medium and removing the bypass a level of 3 μg/litre was measured after two hours. One may thus assume that the agent was incorporated into the silicone tubes of the circulation model and was released again therefrom in the sense of an equilibrium system.

The experiments were also performed for sirolimus. A concentration of 1.3 μg/cm² was measured in the prosthesis. There was a rapid increase in the medium to 7 ng/ml, the maximum was 9 ng/ml.

Ex-vivo trial: Such a prosthesis was anastomosed with a bovine artery. The IS content was determined in the tissue of the adjacent bovine artery after 24 hours. A direct dependency on the distance could be shown in connection therewith. Immediate to the anastomosis the value was >30 μg/l/mm², at a distance of 3 mm from the anastomosis the value was 22.8 μg/l/mm², at a distance of 5 mm the value was 14.2 μg/l/mm² and no values could be obtained more far away. This experiment evidenced the elution into organic tissue.

### EXAMPLE 6

### Elution of Molecules from the Prosthesis—Studies in the Animal Model

In a last step the prostheses were implanted in a preliminary animal trial in order to show the impact on intimal hyperplasia. Six mini-pigs of the Gottingen type were used as test animals. The Aa iliacae was prepared by a transperitonial access under general i.v. anaesthesia (Thiopental®) and each a multifunctional and a standard prosthesis were introduced into every animal. All animals obtained an antibiotic prophylaxis (Spizef®) and were anticoagulized with heparin.

As medicaments each of paclitaxel, sirolimus and IS was used twice. We have applied patch plasties as the border line between prosthesis and artery is bigger compared to end-to-end anastomoses and we could thus assess intimal hyperplasia in cross-section very well. The animals were again subjected to surgery after six weeks and the iliac bifurcation was explanted. Samples in longitudinal direction were derived from the patch plastics in the proximal and in the distal region, respectively, and samples in transversal direction were derived from the medium region. These samples were fixed in formaldehyde. The further processing comprised dehydration in an increasing alcohol series until embedding into paraffin. The preparations were cut and stained in HE and Tri-PAS. Apart from the extent of IH, also integration and inflammatory reactions were assessed.

All surgeries could be performed without any incidents, in all cases patch plastics had been applied to the iliac bifurcations. In the further course there were no exceptional findings, all animals recovered quickly from surgery. From a clinical point of view there were no signs of ischaemia in any of the animals.

The animals were sacrificed after 6 weeks and the iliac bifurcation explanted. All prostheses were well integrated into the retroperitoneum. There was no indication for delayed wound healing or infection as depicted in FIG. 9, whereby FIG. 9 a) shows the good integration of the prosthesis of the invention at the explanted iliac bifurcation, and FIG. 9 b) shows the histology thereof.

There were also no differences in integration behaviour in terms of histology between the conventional and the new, silicone coated, medicament bearing prostheses. Foreign body reactions were still observed for all of the prostheses.

A reduction in intimal hyperplasia could be shown for the materials coated with immunosuppressive agent. This effect was observed for both IS and sirolimus and is depicted in FIG. 10. FIG. 10 a) and FIG. 10 b) show that conventional prostheses where the border wall between patch and artery (a) can be recognized which corresponds to intimal hyperplasia (b). FIGS. 10 c) and d) show the prosthesis of the present invention, whereby there is no border wall and there is only little intimal hyperplasia observed in the histologic representation depicted in FIG. 10 d).

Using paclitaxel a complete suppression of endothelialization could be observed, whereby the patch was well integrated from outside as may be taken from FIG. 11.

The features of the present invention disclosed in the preceding specification, the claims and the figures can, individually as well as in any combination, be essential for the practice of the invention in its various embodiments.

1. A prosthesis comprising a proximal and a distal end and a lumen extending in between, whereby the prosthesis comprises a support structure and at least one biologically active compound.

2. The prosthesis according to claim 1, characterized in that the support structure consists of a support material and a coating material.

3. The prosthesis according to claim 2, characterized in that the support material is selected from the group comprising polytetrafluoroethylene, polyester and polyurethane.

4. The prosthesis according to claim 2 or 3, characterized in that the coating material is selected from the group-comprising organopolysiloxanes.

5. The prosthesis according to claim 4, characterized in that the organopolysiloxane is selected from the group comprising polydimethylsiloxane, polyvinylsiloxane, polyphenylsiloxane and polyalkylsiloxane.

6. The prosthesis according to any of claims 1 to 5, characterized in that the support material and the coating material form a matrix.

7. The prosthesis according to claim 6, characterized in that the matrix contains the biologically active compound.

8. The prosthesis according to any of claims 1 to 7, characterized in that the support structure defines an outer wall and an inner wall, whereby the inner wall limits the lumen conducting the blood, and the outer wall limits the support structure against the surrounding tissue.

9. The prosthesis according to claim 8, characterized in that the prosthesis is liquid tight.

10. The prosthesis according to any of claims 5 to 9, characterized in that the coating material is polydimethylsiloxane and preferably the support material is polyester, whereby the coating of the prosthesis is 10-30 mg polydimethylsiloxane/cm² surface, preferably 15-26 mg polydimethylsiloxane/cm² surface and most preferably 16-23 mg polydimethylsiloxane per cm² surface.

11. The prosthesis according to any of claims 1 to 10, characterized in that the coating material comprises a modification.

12. The prosthesis according to claim 11, characterized in that the modification is selected from the group comprising silanolate, alkyl hydroxide, polyvinyl alcohol, polyethylene oxide, zinc sulfate, polyvinyl pyrrolidone, and phosphocholine.

13. The prosthesis according to claim 12, characterized in that the modification is selected from the group comprising polyvinyl alcohol, polyvinyl pyrrolidone and phosphocholine.

14. The prosthesis according to any of claims 11 to 13, characterized in that the modification is present at the inner wall and preferably remains there permanently.

15. The prosthesis according to any of claims 1 to 14, characterized in that at least one biologically active compound is present in the matrix.

16. The prosthesis according to any of claims 1 to 15, characterized in that the biologically active compound is selected from the group comprising antibiotics, immunosuppressive agents and proliferation inhibitors.

17. The prosthesis according to any of claims 1 to 16, characterized in that the biologically active compound is selected from the group comprising acetylsalicylic acid, sirolimus and paclitaxel.

18. The prosthesis according to any of claims 1 to 17, in particular according to any of claims 10 to 17, characterized in that the biologically active compound is released at a rate of 2-3 μg/hour.

19. The prosthesis according to any of claims 1 to 18, characterized in that the prosthesis is a vascular prosthesis.

20. The prosthesis according to any of claims 1 to 19, characterized in that the prosthesis is used in alloplastic below or above knee reconstruction

21. The prosthesis according to any of claims 1 to 19, characterized in that the prosthesis is used as a peripheral or central AV shunt.

22. The prosthesis according to any of the preceding claims, characterized in that the prosthesis is used as a patch or as a dialysis shunt.

23. A method for the manufacture of a prosthesis comprising a proximal and a distal end and a lumen extending in between, whereby the prosthesis comprises a support structure and at least one biologically active compound and the support structure consists of a support material and a coating material, preferably a prosthesis according to any of claims 1 to 22, comprising the steps of:
providing the support material, whereby the support material comprises a proximal and a distal end and a lumen extending in between;
coating the support material with a coating material, whereby a matrix is formed;
introducing the biologically active compound into the matrix.

24. The method according to claim 23, characterized in that the coating of the support material is performed by applying the coating material onto the rotating support material.

25. The method according to claim 23 or 24, characterized in that the coating step is repeated several times and, preferably, the applied coating material or the coated support material is dried after each coating step.

26. The method according to any of claims 23 to 25, characterized in that the coating material already comprises a modification.

27. The method according to any of claims 23 to 25, characterized in that the coating material and/or the matrix is provided with a modification, preferably on the surface, and more preferably at the wall of the support structure facing the lumen, and is modified therewith.

28. The method according to claim 27, characterized in that the modification is performed by a wet-chemical modification method.

29. The method according to any of claims 23 to 28, characterized in that the modification is formed by a molecule or a group provided thereby which is selected from the group comprising polyvinyl alcohol, phosphocholine and polyvinyl pyrrolidone.

30. The method according to claim 29, characterized in that prior to the wet-chemical modification method the matrix is activated by means of an alkaline methanol solution.

31. The method according to claim 30, characterized in that after the activation the modification generating molecule is contacted with the matrix in a diluted solution, preferably contacted with the inner wall of the matrix and/or the support structure.

32. The method according to any of claims 23 to 31, characterized in that the biologically active compound is introduced into the matrix by a wet-chemical method.

33. The method according to claim 32, characterized in that prior or during the introduction of the biologically active compound the matrix is swollen by contacting the matrix with a swelling agent.

34. The method according to claim 32 or 33, characterized in that the biologically active compound is provided in a solvent and that the biologically active compound containing solvent is contacted with the matrix.

35. The method according to claim 34, characterized in that the solvent is a swelling agent for the matrix.

36. The method according to any of claims 24 to 35, characterized in that the support material is rotated along its longitudinal axis which extends through or is parallel to the lumen of the support material while the coating material is applied to the support material.

37. The method according to any of claims 23 to 36, characterized in that the coating material is provided as a prepolymer and is polymerized during or after the contacting with the support material.

38. The method according to any of claims 36 to 37, characterized in that the coating material is applied from the distal end to the proximal end or from the proximal end to the distal end, whereby the support material is moved relative to the applied coating material in the direction of the longitudinal-axis of the support material.

39. The method according to any of claims 23 to 38, characterized in that the support material consists of a polyester and that the coating material is polydimethylsiloxane.

40. The method according to claim 39, characterized in that the support material is rotated at 80 revolutions/minute.

41. The method according to claim 40, characterized in that the coating material is a solution of 11 g of the pre-polymer of polydimethylsiloxane in 86 g ethylacetate.

42. The method according to claim 41, characterized in that the relative movement is 14 mm/minute and the dripping rate is 15-20 ml/hour.

43. The method according to any of claims 35 to 42, characterized in that a coating cycle is completed once the support material has been coated from its proximal end to its distal end or from its distal end to its proximal end.

44. The method according to claim 43, characterized in that the coating cycle is repeated several times.

45. Prosthesis obtainable by a method according to any of claims 23 to 44.
